(19) United States
(12) Patent Application Publication
(10) Pub. No.: US 2010/0106029 A1
(43) Pub. Date: Apr. 29, 2010

(54) ARTERIAL BLOOD PRESSURE MONITOR WITH A LIQUID FILLED CUFF

(75) Inventor: Jacob Fraden, San Diego, CA (US)

Correspondence Address: DARBY & DARBY P.C., P.O. BOX 770, Church Street Station, New York, NY, 10008-0770, US

(73) Assignee: KAZ, INCORPORATED, Southborough, MA (US)

(21) Appl. No.: 12/532,812

(22) PCT Filed: Feb. 19, 2008
(86) PCT No.: PCT/US08/54299

§ 371 Date: Sep. 23, 2009

Related U.S. Application Data
(60) Provisional application No. 60/920,733, filed on Mar. 28, 2007.

(51) Int. Cl. A61B 5/022

(52) U.S. Cl. 600/493; 600/490; 600/499

(57) ABSTRACT

A non-invasive arterial blood pressure monitor uses an inflatable cuff that incorporates the first bladder that is filled with non-compressible liquid or gel. The bladder can be pressurized by an action of a pressurizing device superimposed onto its outer surface. In a preferred embodiment, a pressurizing device is an air-filled second bladder being connected to an air pump and bleed valve. The first bladder is positioned between the patient's body and the second bladder. During operation, the second bladder compresses the first bladder, which, in turn, compresses the patient's artery against the supporting bone. The mechanical coupling between the blood-filled artery of a patient and the liquid-filled bladder of a dual-bladder cuff is improved for detecting pressure oscillations in a broad frequency range. The pressure sensor that is coupled to the first bladder also functions as a hydrophone for picking-up the mechanical oscillations from any part of the occluded limb or digit. This allows for improved computation of the arterial pressure.

This patent claims the benefit of U.S. Provisional Patent Application No. 60/920,733 filed on Mar. 28, 2007, which is incorporated by reference herein.

### BACKGROUND OF THE INVENTION

1. Field of the Invention

This invention relates generally to methods and medical apparatuses for non-invasive monitoring of arterial blood pressure, and specifically to the devices and methods that use inflatable cuffs.

2. Description of Related Art

Blood pressure monitoring has rapidly become an accepted and, in many cases, essential aspect of human and veterinary treatment. Such monitors are now a conventional part of the patient environment in emergency rooms, intensive and critical care units, and in the operating theatre, as well as in homes.

Four well known techniques have been used to non-invasively monitor a subject's arterial blood pressure waveform, namely, auscultation, oscillometric, tonometry and flowmetry. The auscultation, oscillometric and flowmetry techniques use a standard inflatable cuff that occludes an artery, e.g., the subject's brachial artery. The auscultatory technique determines the subject's systolic and diastolic pressures by monitoring certain Korotkoff sounds that occur as the cuff is slowly deflated or inflated. The oscillometric technique, on the other hand, determines these pressures, as well as the subject's mean pressure, by measuring the small pressure oscillations that occur in the cuff as the cuff is deflated or inflated. The flowmetric technique relies on detecting the variations in blood flow downstream from the cuff.

The oscillometric method of measuring blood pressure is the most popular method in commercially available automatic systems. This method relies on measuring changes in arterial counter pressure, such as imposed by an inflatable cuff, which is controllably relaxed or inflated. In some cases the cuff pressure change is continuous, and in others it is incremental. In substantially all oscillometric systems, a transducer (pressure sensor) monitors arterial counter pressure oscillations, and the processing electronics converts select parameters of these oscillations into blood pressure data.

In the oscillometric method, the mean blood pressure value is the mean of the cuff pressure that corresponds in time to the peak of the envelope of the pressure oscillations. Systolic blood pressure is generally estimated as pressure of the decaying pressure slope prior to the peak of the pressure oscillations envelope that corresponds in time to where the amplitude of the envelope is equal to a fraction of the peak amplitude. Generally, systolic blood pressure is the pressure on the decaying pressure of the cuff prior to the peak of the envelope where the amplitude of the envelope is 0.57 to 0.45 of the peak amplitude. Similarly, diastolic blood pressure is the pressure on the decaying pressure of the cuff after the peak of the envelope that corresponds in time to where the amplitude of the envelope is equal to a different fraction of the peak amplitude. Often, diastolic blood pressure is conventionally estimated as the pressure on the decaying pressure of the cuff after the peak where the amplitude of the envelope is equal to 0.82 to 0.74 of the peak amplitude. Other algorithms are also known in the art.

The auscultatory method also involves inflation of a cuff placed around a cooperating artery of the patient. Systolic pressure is indicated when Korotkoff sounds disappear as the cuff is inflated above the highest pressures exerted by the heart onto the arterial walls. Diastolic pressure is indicated when the Korotkoff sounds first appear when the cuff pressure is elevated above the atmospheric pressure. The auscultatory method can only be used to determine systolic and diastolic pressures, and it does not determine mean pressure.

Often, both the oscillometric and auscultatory methods lack accuracy and consistency for determining systolic and diastolic pressure values. The oscillometric method applies an arbitrary ratio to determine systolic and diastolic pressure values. As a result, the oscillometric method sometimes does not produce blood pressure values that agree with the more direct and generally more accurate blood pressure values obtained from the arterial-line method (e.g., catheter inserted). Furthermore, because the oscillating signal amplitudes detected from the cuff are very low compared to the mean pressure of the cuff, even a small amount of noise can result in inaccurately measured blood pressure values. Similarly, the auscultatory method requires a judgment to be made as to when the Korotkoff sounds start and when they stop. This detection is made when the Korotkoff sound is at its very lowest. This is especially apparent due to traveling of the acoustic waves through media of different densities, such as biological tissues, air, inflatable bladder, cuff fabric, microphone components, etc. As a result, both the oscillometric and auscultatory methods are subject to inaccuracies due to low signal-to-noise ratios.

The third method used to determine arterial blood pressure has been tonometry. The tonometric method typically involves a transducer including an array of pressure sensitive elements positioned over a superficial artery. Hold-down forces are applied to the transducer so as to flatten the wall of the underlying artery without occluding the artery. The blood pressure is sensed by measuring forces exerted by blood pressure pulses in a direction perpendicular to the underlying artery.

The fourth method used to determine the arterial blood pressure has been the flowmetric method. This method relies on detecting changes in the blood flow downstream from the cuff and relating such changes to the cuff pressure.

As an alternative to measuring arterial pressure in the brachial artery (upper arm), the forearm (below the elbow) and the wrist monitors are also feasible and practical measurement sites. They rely primarily on occlusion of the radial and ulnar arteries. Since blood flow in these arteries is smaller than that in the brachial artery, the signal-to-noise ratio decreases even further. Another problem with the wrist cuffs is difficulty in occluding the radial and ulnar arteries against the radius and ulnar bones since these bones are rather small to serve as a reliable support for compression of the arteries. Furthermore, numerous tendons near the wrist arteries may interfere with the occlusion resulting in a poor mechanical coupling between the cuff and arteries.

When Korotkoff sounds are used in determining the systolic and diastolic pressures, it is imperative to position the microphone directly over the monitored artery, otherwise a signal-to-noise ratio is reduced even more and accuracy will be greatly compromised. To resolve the error problems that arise from the above factors, numerous cuff designs have been proposed. Among these is the use of a liquid-filled invasive cuff applied directly to the artery as described in U.S. Pat. No. 4,256,094 issued to Kapp et. al. which is incorporated by reference herein. U.S. Pat. No. 3,527,204 issued to Lem which is incorporated by reference herein teaches a dual cuff where the liquid-filled chamber is positioned on the top of the air-filled chamber and the pressure exerted over the patient's limb is developed by applying pressure to both air and liquid. A dual-cuff design with side-by-side bladders is described in U.S. Pat. No. 3,752,148 issued to Schmalzbach which is incorporated by reference herein. A dual air chamber design with two chambers positioned in layers is disclosed in U.S. Pat. No. 7,250,030 which is incorporated by reference herein. A semi-rigid outer layer on the outside surface of a cuff is described in U.S. Pat. No. 6,224,558 issued to Clemmons which is incorporated by reference herein. U.S. Pat. No. 7,250,030 issued to Sano et al. describes a dual-chamber bag where both chambers are filled with the same type of fluid. These and other designs have failed to solve many accuracy problems, henceforth further improvements of the cuff system are needed.

### SUMMARY OF THE INVENTION

The present invention is directed to an inflatable cuff that incorporates a first chamber (bladder) that can be compressed against the patient limb by a pressurizing device superimposed onto its outer surface. In one embodiment, the pressurizing device includes a second chamber filled with gas or air, and is referred to as the AFC (Air-Filled Chamber). The first chamber is positioned between the patient's arm, forearm, wrist or digit and the AFC. The first chamber is filled with a non-compressible substance such as liquid or gel and may be coupled to a pressure sensor and, in some embodiments, to a microphone that picks-up the Korotkoff sounds. The first chamber is referred to as the LFC (Liquid-Filled Chamber). The density of the liquid or gel is relatively close to that of blood. During operation, the AFC compresses the LFC which in turn, compresses the artery against the supporting bone. The mechanical coupling between the blood-filled artery and the LFC of a dual-chamber cuff is improved. Since the LFC to a large degree circumferences the arm or wrist, positioning of the cuff becomes less critical.

### BRIEF DESCRIPTION OF DRAWINGS

Further details and aspects of example embodiments of the present invention are described in more detail with reference to the Figures, in which:

FIG. 1 is an illustration of a human limb with an arterial pressure monitor positioned over the upper arm;

FIG. 2 is a diagram of a dual-chamber cuff with the air pump and pressure transducer being coupled to the air-filled chamber (AFC);

FIG. 3 is an illustration of a dual-chamber cuff with the liquid pump and pressure transducer being coupled to the liquid-filled chamber (LFC);

FIG. 4 is a diagram of a dual-chamber cuff with the pump connected to the AFC, while the pressure transducer and microphone are being coupled to the LFC;

FIG. 5 is a graph of the relationship between cuff pressure and Korotkoff sounds;

FIG. 6 is a diagram of the monitor with a separate microphone;

FIG. 7 is a graph of the cuff pressure increase used to find the systolic pressure level;

FIG. 8 is an illustration of an arm cuff with a finger plethysmographic sensor;

FIG. 9 is a diagram of a finger photo-plethysmographic sensor;

FIG. 10 is a graph of the relationship between the cuff pressure oscillations and output signals of a finger photo-plethysmographic sensor;

FIG. 11 is a graph of the relationship between the oscillations and Korotkoff sounds;

FIG. 12 is a cross section view of the combination of the air and liquid filled chambers with the liquid filled chamber having a variable wall thickness;

FIG. 13 is a cross sectional view of two cuff chambers having a common wall;

FIG. 14 is a cross sectional view of a liquid filled chamber with a thicker outer wall;

FIG. 15 is a cross-sectional view of a dual chamber cuff with the air filled chamber having peripheral splits;

FIG. 16 is a block diagram of the electrical connection of the inclination sensor;

FIG. 17 is an illustration of the inclination sensor in a tilted position;

FIG. 18 is a diagram of the blood pressure monitor with the air holding tank;

FIG. 19 is a perspective view of a resilient bladder in the form of a plate;

FIG. 20 is a perspective view of a resilient bladder in a pre-formed shape;

FIG. 21 is a cross sectional view of a liquid filled bag with the thick peripheral walls of the envelope and

FIG. 22 is a block diagram of a wireless coupling between the cuff and display.

### PREFERRED EMBODIMENTS

This invention relates to arterial blood pressure noninvasive measurement methods which may include, for example, the oscillometric, auscultatory, and flowmetric methods. All these methods employ pressurizing cuffs. The oscillometric method relies on analyzing oscillations of the cuff pressure, while the auscultatory method is based on analyzing the acoustic waves (Korotkoff sounds) produced inside the compressed artery. A combination of the two systems potentially can be used to improve accuracy.

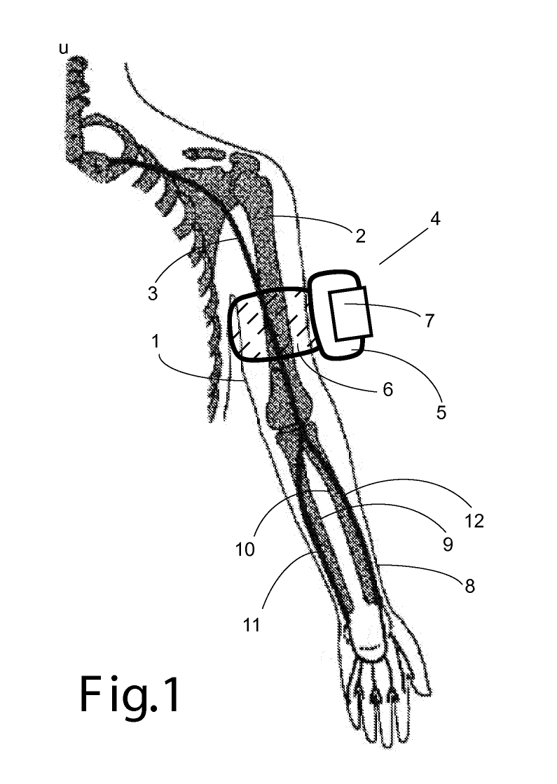

FIG. 1 illustrates an arterial blood pressure monitor 4 with an inflatable cuff 6 wrapped around the upper arm 1 of a patient. The monitor 4 includes an electronic module 5 with a display 7. The cuff 6 may be inflated and thus compress the brachial artery 3 against the supporting humerus bone 2, causing a restriction of the blood flow inside the artery. The cuff 6 contains at least one chamber (bladder) that may be filled with fluid—either gas or liquid. In the prior art devices, air has been commonly employed as the bladder filling fluid.

Alternatively, the cuff 6 may be positioned over the forearm or wrist to compress the radial 12 and/or ulnar 11 arteries against the radius 10 or ulnar 9 bones, respectively. An aspect of this invention is the use of a non-compressible liquid inside the chamber that is part of the cuff. Pressure is applied to the liquid filled chamber which in turn is applied against the patient's arm. This requires the use of a pressurizing device that exerts pressure onto the liquid inside the chamber and subsequently onto the artery in the patient's limb or digit. The pressurizing device may include various components, such as AFC, air pump, hoses, etc. These components may be directly attached to the LFC or can be positioned externally to LFC, depending on their specific functions and purpose. There are numerous ways of designing the pressurizing device as described below.

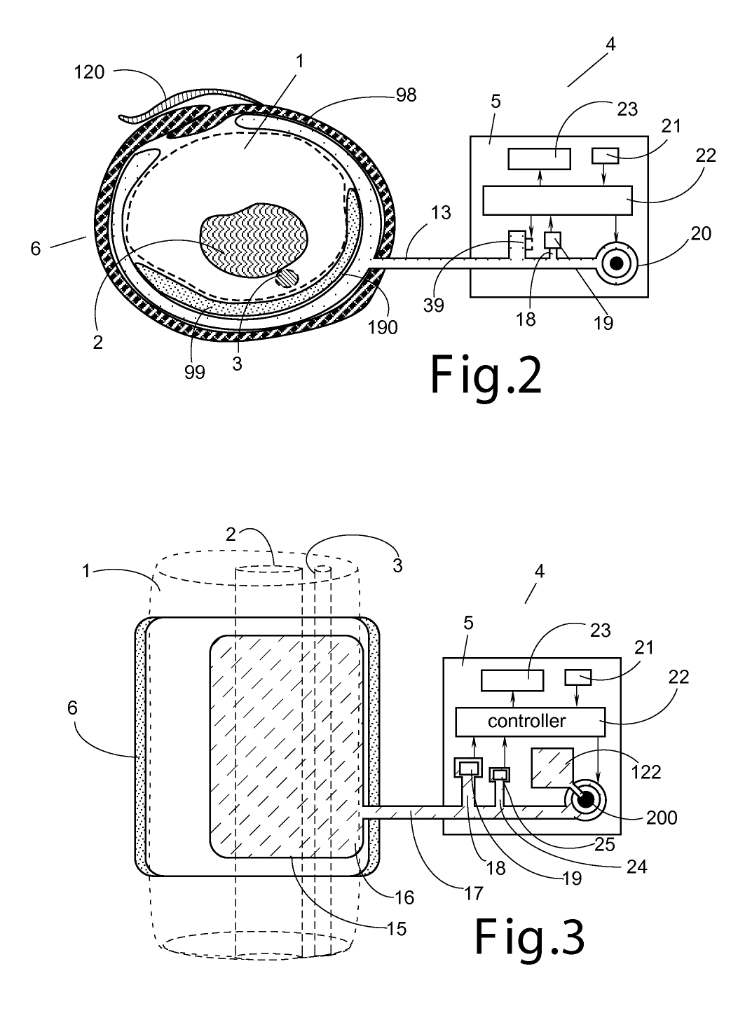

FIG. 2 illustrates a first embodiment of a blood pressure monitor according to the present invention, which includes a second chamber (bladder) LFC 99 positioned between the AFC 98 (part of the pressurizing device) and the patient limb 1 or digit. Both chambers are supported by the cuff 6, which may be made of a thin pliant fabric or plastic material. The LFC need not be the same size as the AFC. In some cases, especially when used with a hydrophone (see below) it may be smaller, but not larger. The cuff wraps around the limb or digit and may be locked in place by a conventional fastener 120, for example VELCRO. The bleed valve 39 (a pressure varying device) may pneumatically connect the AFC 98 to the atmosphere as determined by the controller 22. Initially, on command from the controller 22, the bleed valve 39 closes and the air pump 20 inflates AFC 98 by pumping air in. The pump serves as part of the pressurization device. The air pressure is measured by the pressure sensor 19 which is coupled to the AFC 98 by a link 18 and hose 13. When the AFC 98 inflates, air pressure inside of it compresses the underlying LFC 99 against the arm 1 and, in turn, compresses the artery 3 against the supporting bone 2. The LFC 99 is filled with a non-compressible substance or filler, such as liquid, for example water or mineral oil, whose density is much closer to that of blood than to that of air. Preferably, the density of the LFC substance should differ from the blood density by no more than 50%. Blood density is about 1060 kg/m³. Water density is 1000 kg/m³, mineral oil is 866 kg/m³. Thus, both water and mineral oil may be used. Alternatively, the LFC may be filled with other substances such as non-compressible aqueous or organic gels. All these materials have densities which are relatively close to the density of blood. Viscosity of the filler material (substance) should be adjusted for a particular cuff design and method of fabrication and typically should be no greater than 2000 cP (centipoise). For example, an LFC with a relatively long tube (e.g., over 50 mm) connected to the hydrophone would require a low viscosity substance—approaching the viscosity of water. A relatively shorter tube (e.g., less than 10 mm) could use a material with a higher viscosity, e.g. a viscosity approaching 2000 cP.

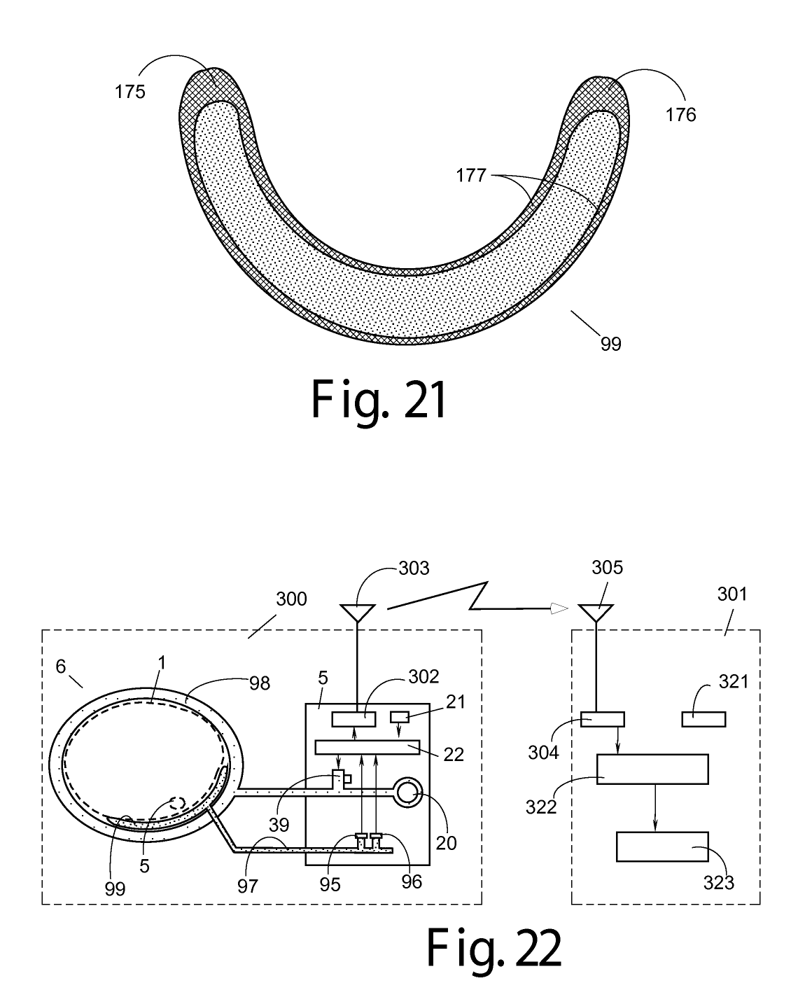

The LFC 99 is sealed and can neither be additionally filled in nor bleed out its liquid or gel. Note that the LFC 99 is fabricated of a pliant and flexible material, like latex or polyethylene, that can conform to the shape of the patient without forming wrinkles in its envelope. Care should be taken to prevent a “bubbling” effect that could be a result of squeezing the filler material to the peripheral sides of the LFC. This can be accomplished by thickening the peripheral portions 175 and 176 (FIG. 21) of the outer envelope 177 of the LFC bladder 99.

Alternatively, the LFC may be molded or otherwise fabricated from a relatively high viscosity resin (over 10,000 cP), as a flat pliant and flexible plate (FIG. 19) where the inner surface 170 is to be positioned toward the patient. This plate will be bent when positioned under the AFC. In another embodiment the LFC may be pre-shaped to conform to the arm or wrist shape (FIG. 20) with the inner surface 171 positioned toward the patient. The material for the embodiments of FIGS. 19 and 20 can be any suitable pliant and resilient material, such as latex or silicone rubber whose densities should be substantially close to that of blood.

Pressure of the liquid inside the LFC 99 (or of the material forming LFC in FIGS. 19 and 20) is uniformly distributed over its entire volume. This causes a conformal and uniform compression of the biological tissues against the supporting bone 2. Since the density of the liquid inside the LFC 99 is close to that of blood in the artery 3, oscillations of the artery are well coupled to the LFC 99 and subsequently coupled to the AFC 98 over the large coupling surface 190 between them. Note that in this embodiment, the LFC is a passive (no sensors attached to it) intermediate medium between the AFC and the artery. The arterial oscillations from the AFC are converted by the air pressure sensor 19 and/or microphone (not shown in FIG. 2) and fed into the controller 22 for signal processing and display.

The second embodiment of FIG. 3 is similar to FIG. 2, except the LFC 15 is not an intermediate medium between the artery and the pressure sensor. Here, the AFC is not employed at all. The LFC 15 is attached by a tube 17 and extensions 18 and 24 to the liquid pressure sensor 19 and hydrophone 25, respectively. A reservoir 122 is connected to the liquid pump 200, which, in turn, is connected to the tube 17, thus forming a closed circuit. Liquid 16 can be pumped from reservoir 122 to LFC 15 and vice versa as directed by controller 22. The LFC 15, when liquid 16 is pumped into it, compresses the artery 3 against the supporting bone 2. This arrangement produces even better signal-to-noise ratio as there is no intermediate air-filled bladder between the hydrophone 25 and the artery 3. Liquid in the LFC 15 may be pressurized by any known liquid pump of a peristaltic or piston type that may be driven by an electric motor. Alternatively, an electrically-activated polymer (EAP) may serve as a pressurizing device.

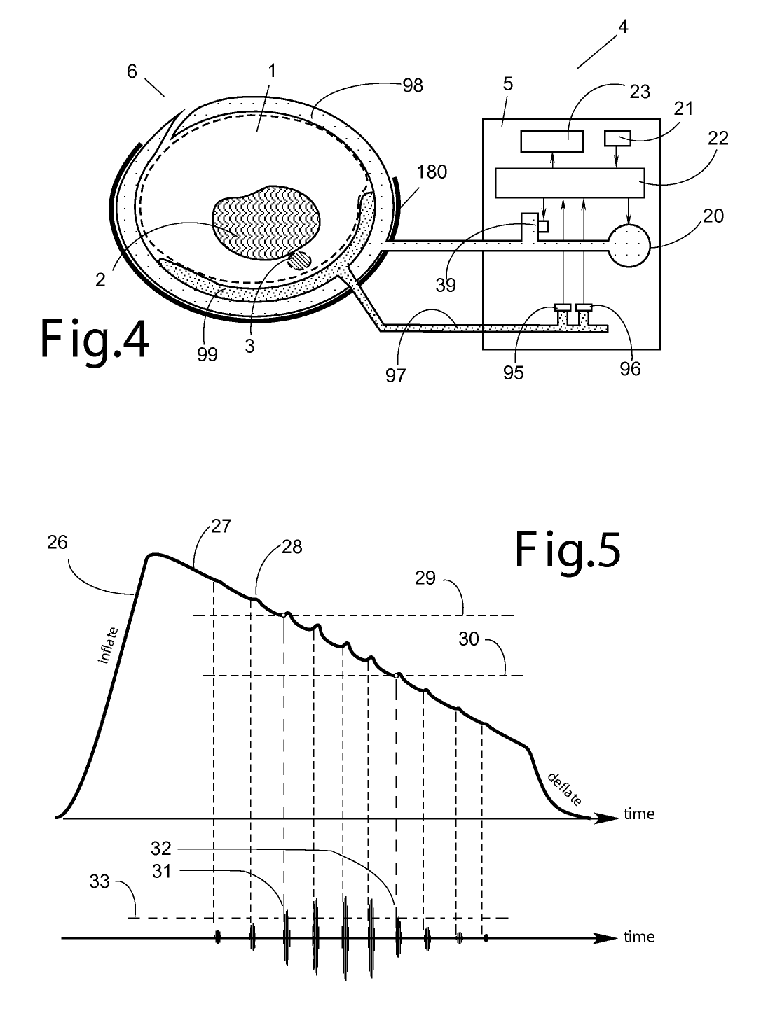

FIG. 4 illustrates another preferred embodiment, where the cuff 6 contains two chambers filled with fluids of substantially different densities. The first chamber is the air filled chamber 98 (AFC) that may or may not entirely encircle the patient's arm 1. Its purpose is to compress the second liquid filled chamber 99 (LFC) against the artery 3. The AFC 98 may be opened to the atmosphere via the bleed valve 39 and can be alternatively inflated from the atmosphere by the air pump 20 when the valve is closed. The pump is part a pressurizing device and the valve is a pressure varying device. An electrically-activated polymer (EAP), e.g., a material which is capable of expanding and exerting pressure when subjected to an electrical current, may serve as a pressurizing device. In this embodiment, the pressure, its oscillations and/or sounds are measured from the LFC, while pressurizing is performed by the AFC or other appropriate structure.

When the AFC 98 inflates, it compresses the LFC 99 against the arm 1. The LFC 99 via a duct 97 is coupled to the hydrophone 95 and liquid pressure sensor 96, both of which are electrically connected to the controller 22. Alternatively, the pressure sensor 96 may be coupled to the AFC 98. As in other embodiments, the liquid in the LFC 99 may be water, oil, or any other non-compressible liquid or gel. A semi-rigid support 180 is superimposed on the outer surface of the cuff to prevent stretching of the AFC 98 outwardly and to maintain direct pressure toward the patient.

A hydrophone 95 as a separate component may not be required in cases when the pressure sensor 96 has a fast speed response. It is not uncommon to employ a pressure sensor with a response rate on the order of 1 ms. Such a fast sensor, in addition to measuring pressures in the LFC, may also act as a hydrophone to pick up both the low and high frequency ranges of the pressure. The pressure sensor spectrum will contain components related to pressure of the liquid, the pressure oscillations and the Korotkoff sounds. These components can be separated either by hardware or software band-pass filters. The signal filters separate these components before further processing. Typically, a low-pass filter having bandwidth approximately from 0 to 1 Hz would pass the LFC's pressure signal. A band-pass filter with a pass band approximately from 0.5 to 20 Hz carves out the pressure oscillations. The third band-pass filter is for passing the higher frequencies of the Korotkoff sound signals and should have a bandwidth approximately from 10 to 200 Hz. The slowest changing components of the pressure signal correspond to the cuff pressure level, the faster changing components of pressure correspond to the arterial oscillations, while the fastest changing components correspond to the Korotkoff sounds. Relating the faster and fastest components to the slowest component can be used as an indicator of the arterial blood pressure (systolic, diastolic and mean).

In any embodiment described herein, the arterial pressure can be measured either during the cuff pressure increase or decrease, depending on the design and employed algorithm. Thus, the bleed valve 39 (if any) and pumps 20 or 200 operate according to a pre-programmed sequence. All electrical devices in the electronic module 5 are electrically connected to the controller 22, which derives its operating power from the power supply 21. The controller 22 computes the arterial blood pressure and outputs the result of a measurement on the display 23. In some embodiments, the electronic module may be physically decoupled from the cuff and linked to it by a cable or wireless device (FIG. 22). In this case, the cuff and other pressure related parts (pump 20, valve 39, etc.) are located in the transmitter unit 300, while the display 323 is in the receiver unit 301 which also contains the receiver 304, receiving antenna 305, processor 322, and power supply 321. A transmitting antenna 303 is coupled to the transmitter 302. Conventional components of the transmitter unit 300, such as a power supply, switches, are not shown in FIG. 22.

The Korotkoff sounds relate to a changing bladder pressure 26 as shown in FIG. 5 where the vertical axes represent pressure. The slowly decaying slope 27 of pressure, causes appearance of both the Korotkoff sounds (bottom portion of FIG. 5) and oscillations 28 of the pressure slope 27. By selecting a threshold 33, two Korotkoff sound packets 31 and 32 can be detected as corresponding to the systolic and diastolic pressures 29 and 30, respectively. Note that the threshold 33 is not necessarily the same for the systolic and diastolic pressure detection.

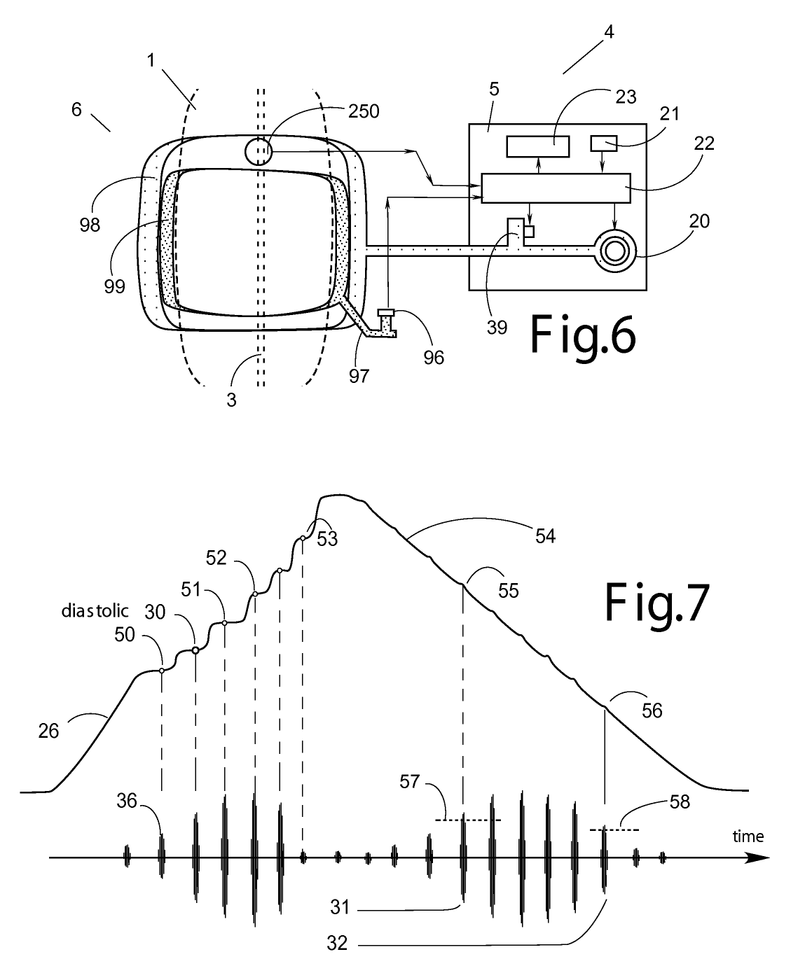

Depending on the actual design of the LFC, the Korotkoff sound amplitude from the LFC may not be sufficiently strong for the signal processing. Thus, a separate microphone 250 (FIG. 6) may be employed. The microphone is positioned outside of the LFC 99 but under the AFC 98. During the measurement, the microphone 250 should be positioned over the cooperating artery 3. Note that the pressure sensor 96 may be positioned closer to the LFC to minimize the noise level.

For the patient's comfort, the maximum pressure exerted on the artery should not be much higher than the systolic pressure. The maximum pressure can be determined from the peak amplitude of the cuff pressure oscillations or the Korotkoff sound as shown in FIG. 7. The pump 20 inflates the cuff in small steps (line 26). When the pump stops at an arbitrary pressure level 50, the Korotkoff sound magnitude 36 is detected. Then, compression continues by a relatively small step, e.g., 20 mm Hg per step, to reach the next pressure level 30 and the Korotkoff sound magnitude is measured again. This continues step-by-step until the cuff pressure 53 is reached when the Korotkoff sound magnitude drops dramatically. This is an indication that the cuff pressure is over both the systolic and diastolic blood pressures. After the pressure 53 is reached, an additional pressure, e.g., 30 mm Hg is added and then a quasi-linear deflations starts. Since every heartbeat corresponds to the appearance of a Korotkoff sound packet, to assure a high measurement resolution, the cuff deflation rate should be between 2 and 5 mm Hg per heart beat. The systolic 57 and diastolic 58 thresholds can be established either as fixed or floating levels to detect the Korotkoff sound packets 31 and 32 so that the controller 22 can relate them to the systolic 55 and diastolic 56 pressures.

Before the threshold comparison is performed, it may be useful to multiply the Korotkoff sounds by the oscillometric pressure fluctuation magnitudes. The multiplication may need to be performed after the Korotkoff sound and pressure oscillations have been subjected to a scaling pre-processing which may include an experimentally determined scaling function. This multiplication will increase the signal-to-noise ratio and improve the threshold comparison. In other words, instead of performing a threshold comparison on each of two signals individually where there may be false threshold detections, the two signals are first multiplied together so that the signal level of the resulting signal is much higher than the noise level of the resulting signal. The product resulting from the multiplication then may need to be multiplied by a scaling factor and compared with a pre-selected fixed or variable threshold. The results of comparison correspond to pressure in the cuff that is measured by the pressure sensor. Another way to improve the noise immunity is by way when the cuff pressure oscillations first being compared with a threshold and then the comparator's output pulses are used as the strobes to gate the Korotkoff sounds before their own threshold comparison. In fact, mathematically a gating is a form of multiplication (multiplying by zero or unity, as in a logical AND function). This is illustrated in FIG. 11 where the pressure oscillations 90 are compared with the threshold 93 to produce pulses 92. These pulses select the Korotkoff sound waves 91 and pass only those sound waves (110) that comply with the AND logical function. Thus, a combination of both the auscultatory and oscillometric signals can improve accuracy and noise immunity.

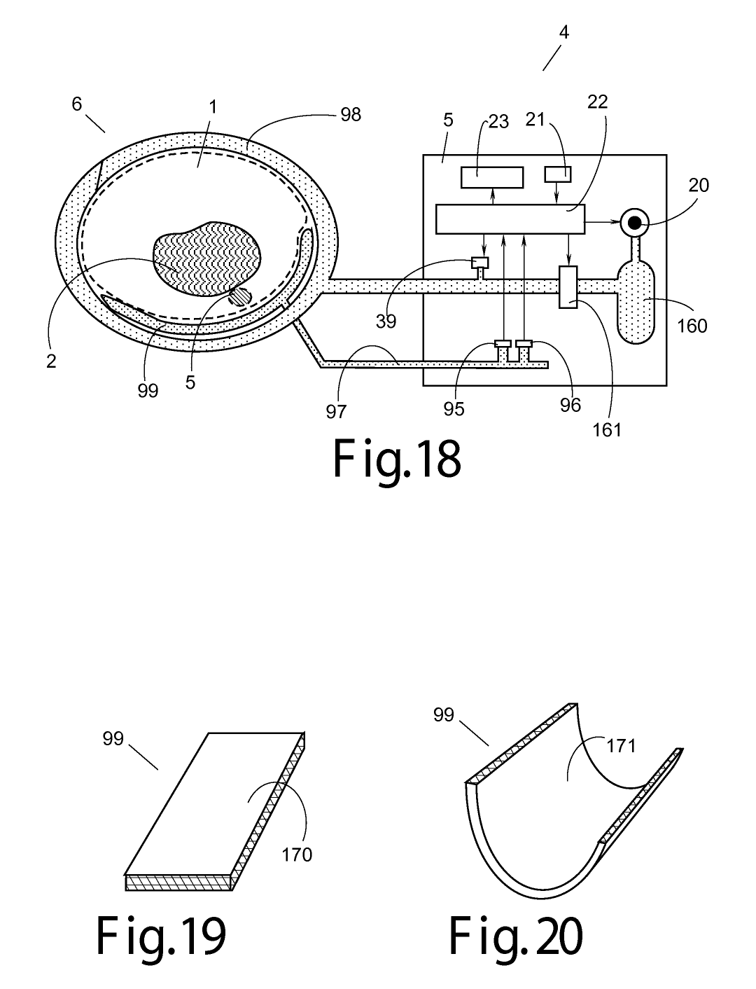

The systolic and diastolic pressures can be detected either on the rising or decaying slopes of the cuff pressure. However, because of the pump noise that is often present during the rising slope (inflation), this may be not practical in all designs. If the circuit of FIG. 4 is employed, the noise level in the LFC 99 may be sufficiently low so the detection can be done on a leading slope. Still, the noise level may be too large for an acceptable accuracy level. One solution to improve performance of the pressurizing device is to use a quiet pump. Another solution (FIG. 18) is to use an air tank 160 that is pressurized by the air pump 20 (or external pump) before the cuff inflation starts. In other words, the tank 160 can be pumped up to a sufficiently high pressure before or between the arterial pressure measurements. The tank holds the pressurized air and releases it into the cuff 98 through the inflation valve 161. A pressurized gas cartridge may serve as a holding tank. The rate of the cuff inflation can be controlled by a pressure varying device such as a variable profile orifice (not shown) inside the inflation valve 161. The orifice size is controlled by the controller 22. Since the pump 20 is turned off during the cuff 98 inflation, the cuff inflation from the air stored in the tank 160 is much quieter. Note that if a pre-pressurized cartridge is employed, no air pump is required.

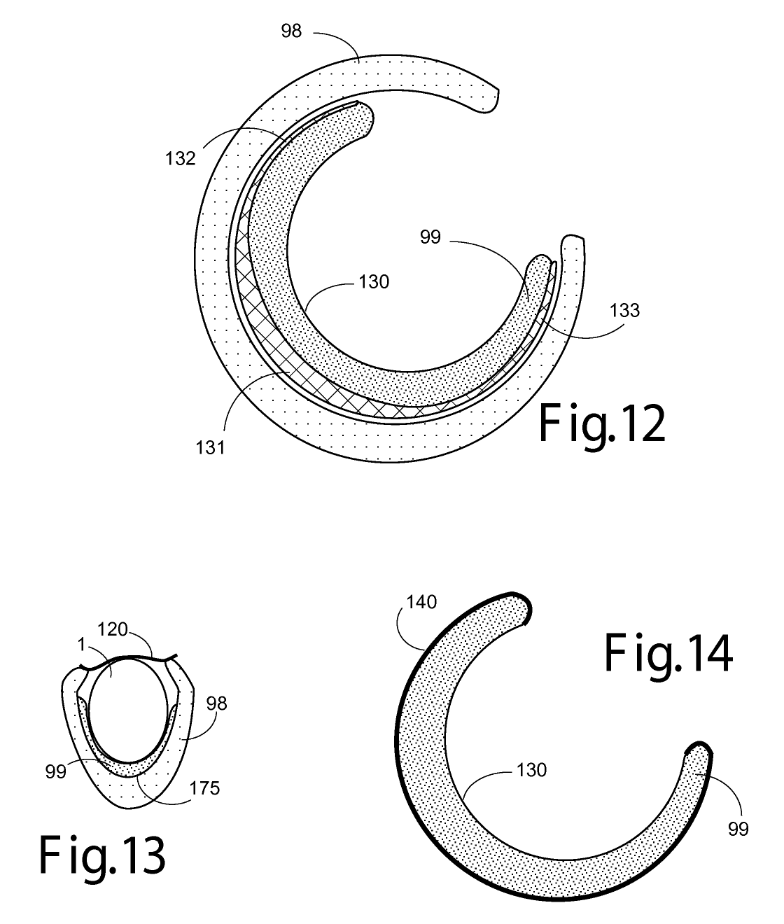

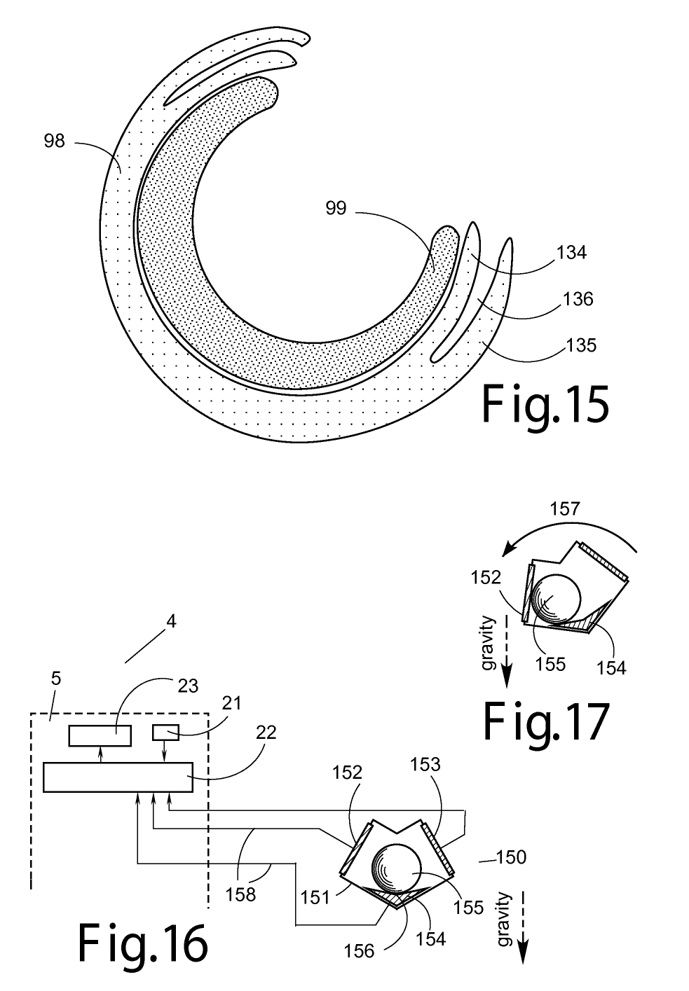

The pressure in the LFC should be distributed uniformly over the entire area of contact with the patient. The support 180 of FIG. 4 helps to direct pressure toward the patient. However, the sides of the LFC may have a reduced elasticity due to the fabricating process or the natural resilience of the LFC envelope. Several techniques may be used to minimize this effect. One technique is illustrated in FIG. 12 which shows the back wall 131 of the LFC 99 having an increased thickness as compared with the peripheral sides 132 and 133. Another solution is shown in FIG. 14 where the back side 140 of the LFC 99 is thicker than the inner side 130 that is coupled (through the cuff fabric) to the patient. Another solution is shown in FIG. 15 where the peripheral sides of the AFC are fabricated in a multi-layer fashion. At the sides, the AFC 98 is separated into at least two thinner extensions, 134 and 135 with a space 136 between them. Note that two chambers (bladders) may be fabricated as a unitary component and share a common wall 175 as in FIG. 13.

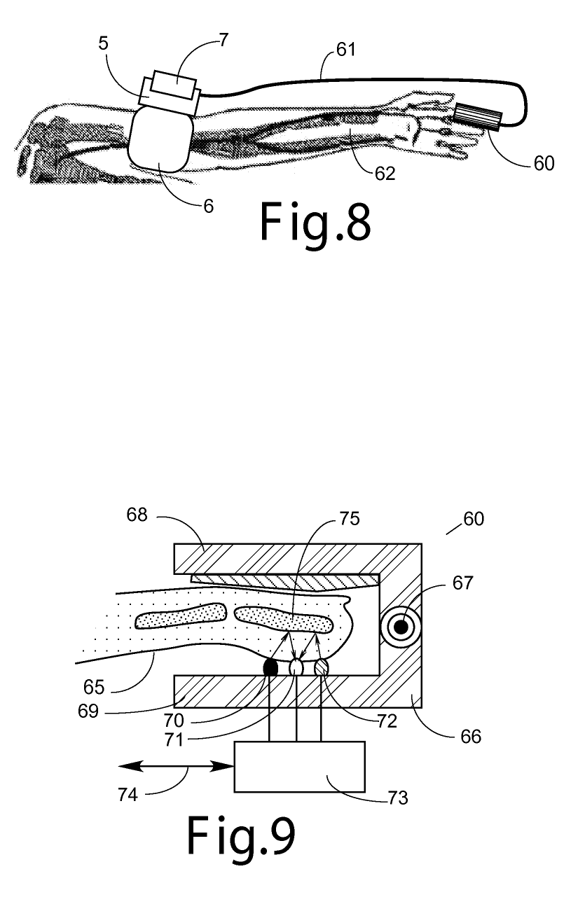

An additional embodiment of the present invention is based on flowmetry, that is, detection of the blood flow in a peripheral artery. When pressure is applied to an artery, it impairs the arterial blood flow. Blood can move freely through the artery only when the externally exerted pressure is less than the diastolic pressure. Pressure above the diastolic but less than systolic reduces the blood flow but does not stop it. When the external pressure exerted onto the artery exceeds the systolic pressure, the artery collapses and the blood flow ceases. This consideration permits using known methods of detecting the arterial blood flow to assess the systolic and diastolic pressures. The known methods of blood flow monitoring are: the Doppler ultrasound and laser monitors, the electromagnetic and thermal diffusion monitors, the reographic and photo-plethysmographic methods and several others. FIG. 8 illustrates an example of a blood pressure monitor with an inflatable cuff of any type described above with a peripheral blood flow sensor (detector) 60 positioned on a digit downstream from the cuff. The sensor 60 is connected to the monitor 5 via a cable 61.

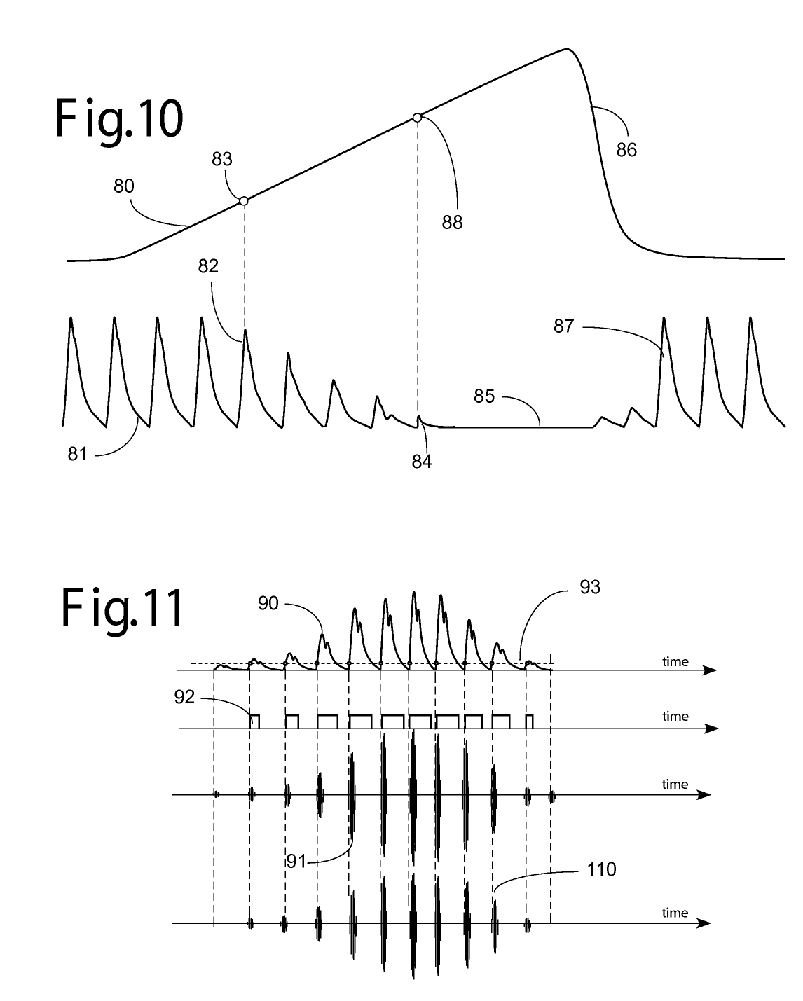

The blood flow sensor 60 of a photo-plethysmographic type as shown in FIG. 9 and includes a photo emitter 70 and a photo detector 71 controlled by the circuit 73 that communicates with the electronic module 5 through the cable 61 using signals 74. The patient's digit 65 (an index finger, e.g.) is inserted into the clamp 66 that may contain two spring-loaded jaws 68 and 69 that are joined by a movable pivot 67. The jaws slightly compress the digit 65 against the photo emitter 70 and the photo detector 71. The light beam emitted by the emitter 70 passes through the skin, fat, capillary blood vessels and bounces off the bone 75. It is returned to the photo detector 71 which converts it to electrical signals. Engorging of the capillary vessels with pulsating blood modulates the light transmission inside the digit. When pressure 80 in the cuff slowly increases (FIG. 10) from zero to the diastolic pressure 83, the light signal 81 waves in the photo detector 71 do not change appreciably. After the diastolic pressure 83 is passed, the light waves 82 start declining until they completely disappear at the level 84 corresponding to the systolic pressure 88. Then, the photo detector 71 will output a baseline signal 85 (no waves). When the cuff is deflated and pressure 86 drops, the light oscillations 87 reappear.

Note that the similar sensor 60 can be also used as a conventional pulse oximeter sensor, but only when the cuff pressure is below the systolic pressure. To detect the blood oxygenation, a second light source (emitter) 72 is added. The wavelengths of the emitters 70 and 72 may be different, e.g., one is red and another is a near-infrared.

Another problem of accuracy relates to a hydrostatic pressure of blood in the arteries. For medical diagnostic purposes, the arterial pressure has to be measured from an artery that is positioned at the level of the aorta. Pressure measured below the aorta level will appear higher while above the aorta it will appear lower. The cause for this is a gravitational force acting on blood. Usually, this is not a serious issue when the cuff is positioned on the upper arm since that position is naturally on the level close to the aorta. However, when the cuff is located at other places, say on a wrist, the patient's wrist usually is situated well below the aorta level, unless the patient is in a supine position. The hydrostatic pressure adds approximately 0.7 mmHg per every centimeter below the aorta level. As a result, when the cuff is positioned on a wrist that rests on a tabletop with the patient sitting at the table, for an average adult person the cuff is located approximately 15 cm lower than the aorta. This will cause the systolic and diastolic pressures to measure about 10 mmHg higher. This error can be compensated to some degree either by elevating the wrist to the aorta level or mathematically by correcting the measured values of the systolic and diastolic pressures. The mathematical correction is an addition of the offsets to the systolic and diastolic pressures. The values of the offsets may be constant or variable, that is controlled by the level of the cuff in relationship to the aorta level.

To detect the position of the cuff various types of position sensors may be employed. One example of the sensor is a tilt (inclination) sensor that may be physically attached to the cuff. The tilt sensor (detector) will measure the degree of the cuff inclination with respect to the gravitational force. This corresponds to elevation of the arm approximately to the aorta level.

Many types of inclination detectors are known in art and can be employed for this purpose. FIG. 16 shows the simplest version of the tilt detector 150, which includes a fully enclosed body 151 fabricated of an electrically insulating material, such as ABS resin. Three electrically conductive contacts 152, 153 and 154 are imbedded into the body 151. At the central contact 154 there is an indentation 156. Inside the body 151, there is a ball 155 that is fabricated of an electrically conductive material, such as nickel plated steel. The ball 155 rests in the indentation 156 when the tilt detector is horizontal as in FIG. 16. Wires 158 connect contacts 152, 153 and 154 to the controller 22. When the cuff is positioned horizontally (the patient's wrist rests on a desk top, e.g.), the ball 155 does not touch contacts 152 and 153. This results in all contacts being insulated from one another and electrical signals in wires 158 indicating to the controller 22 the horizontal position of the cuff. The controller 22 either shows on display 23 that a re-positioning of the wrist is required (raise the wrist to the heart level), or alternatively, makes a mathematical correction by adding the offsets to the measured systolic and diastolic pressures. The values of the offsets may be found experimentally and typically are between −6 and −12 mmHg (negative offsets are typical).

If the cuff and the attached tilt detector 150 rotate in direction 157 (FIG. 17), it is an indication that the patient's wrist is being elevated. A sufficient angle of rotation causes the ball 155 to roll from the central position, shorting contacts 152 and 154. This indicates to the controller 22 that the wrist is in a correct (elevated) position and no correction of the measured arterial pressure is required. Naturally, the simplest tilt detector 150 shown in FIGS. 16 and 17 has only two states: the correct position and the incorrect position of the wrist. A more complex tilt detector that measures the actual degree of inclination may provide a more accurate correction of the measured arterial pressure by varying the magnitude of the offset. Then, the controller can generate a set of numbers by adding or subtracting the offsets from the measured arterial pressure (both systolic and diastolic). These corrected blood pressure numbers may be displayed on a display 23. However, for many practical cases, the simplest version shown in FIG. 16 is usually sufficient.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. A monitoring system for noninvasive measurement of the arterial blood pressure from a body surface of a human or animal patient, comprising
a pressurizing cuff having outer and inner surfaces and containing a first chamber made of a pliant material and adapted to be placed adjacent a body surface of a patient at the inner surface of said cuff, such first chamber including a first substance having a density that is substantially comparable to a density of blood;
a pressurizing device compressing said first substance;
a pressure transducer for producing signals representative of pressure of said first substance;
an electronic module operable to analyze signals received from said pressure transducer and determine arterial blood pressure, and
a display for presenting information corresponding to the arterial blood pressure.

2. The monitoring system of claim 1 wherein said pressurizing device is a second chamber made of a pliant material and being adjacent to the first chamber such that both chambers are disposed in layers over the body surface of a patient, wherein said second chamber is positioned at said inner surface and holds a second substance having a lower density than the density of the first substance.

3. The monitoring system of claim 2 wherein the first substance includes non-compressible fluid and the second substance includes gas;

4. The monitoring system of claim 1 further comprising a pressure changing device for varying pressure within said first substance or said second substance.

5. The monitoring system of claim 1 further comprising a holding tank connected to said pressurizing device.

6. The monitoring system of claim 1 wherein said first substance includes liquid or gel.

7. The monitoring system of claim 1 further comprising a hydrophone coupled to the first chamber and said electronic module.

8. The monitoring system of claim 1 wherein said electronic module contains at least two signal filters, with the first filter being a low pass filter passing frequencies below 1 Hz and the second filter being a band-pass filter passing signals in the frequency range higher than 0.5 Hz.

9. The monitoring system of claim 1 further comprising a microphone positioned adjacent said first chamber.

10. The monitoring system of claim 1 wherein said first chamber is made of a pliant material having a variable thickness.

11. The monitoring system of claim 1 further comprising a semi-rigid support disposed over the outer surface of said inflatable cuff.

12. The monitoring system of claim 1 further comprising a blood flow detector connected to said electronic module.

13. The monitoring system of claim 11 wherein said blood flow detector contains a first light emitting source and a first light sensor.

14. The monitoring system of claim 12 further comprising a second light emitting source producing light in a different spectral range from said first light emitting source.

15. A method of determining arterial blood pressure generated by the heart of a patient body by using an inflatable cuff comprising the steps of
varying pressure inside said inflatable cuff;
measuring pressure inside said inflatable cuff;
generating values of the measured blood pressure in the cuff that correspond to systolic and diastolic pressures;
generating corrected information representative of the arterial blood pressure by applying a respective offset to each said value, wherein said offsets relate to position of the cuff relative to the patient body, and
outputting said corrected information.

16. The method of determining arterial blood pressure of claim 15 further comprising the steps of
measuring a position of the cuff with respect to a position of the heart;
determining said offsets based on the measured position.

17. The method of determining arterial blood pressure of claim 16 where measuring position of the cuff includes determining inclination of the cuff with respect to gravitational force.

18. A method of measuring the arterial blood pressure of a patient body by using a pressurizing cuff comprising the steps of:
wrapping the cuff around a part of the patient body;
changing pressure in the cuff;
measuring a first value of pressure in the cuff;
detecting oscillations in the measured cuff pressure and producing a first signal corresponding to the cuff pressure oscillations;
detecting Korotkoff sounds in the cuff and producing a second signal corresponding to the Korotkoff sounds magnitude;
multiplying the first and second signals to produce a product;
relating the product to the measured pressure in the cuff to determine systolic or diastolic pressure values, and
providing the systolic or diastolic pressure value to an output device.

19. The method of measuring the arterial blood pressure of claim 18 further comprising the step of comparing the product with at least one threshold level;

20. The method of measuring the arterial blood pressure of claim 19, where said threshold level relates to either said first or second signal.

21. The method of measuring the arterial blood pressure of claim 18, further comprising the step of multiplying said product by an experimentally selected scaling factor.

22. A method of determining arterial blood pressure from an external surface of a patient body by using a pressurizing cuff containing two pliant bladders filled with fluids having different densities, comprising the steps of
disposing the second bladder containing fluid of a lower density over the first bladder containing fluid of a higher density;
positioning the first bladder to be adjacent to a surface of the patient body;
varying pressure of fluid of either the higher or lower density;
measuring pressure of the fluid of either the higher or lower density;
relating slow changing components of the measured pressure to fast changing components of the measured pressure;
computing arterial pressure of the patient;
outputting arterial pressure of the patient.

23. The method of determining arterial blood pressure of claim 22 further comprising the step of using wireless transmission of data from said cuff.

24. A pressurizing cuff for applying pressure to a body and detecting signals from the body, the cuff comprising:
a first chamber containing a substantially non-compressible material, the first chamber having a first side positioned adjacent the body and a second, opposite side;
a pressurizing device being positioned adjacent the second side of the first chamber, such that said first chamber is positioned between the body and said pressurizing device;
wherein the pressurizing device is selectively pressurized and depressurized such that pressure is selectively applied by the pressurizing device to the first chamber, which in turn applies pressure to the body.

25. The cuff of claim 24, wherein the pressurizing device includes a second chamber containing a gas.

26. The cuff of claim 24, wherein the body is a human body and application of pressure to the body acts to compress an artery within the body.

27. The cuff of claim 24, wherein application of pressure to the body provides mechanical coupling of signals from the body to the first chamber.

28. The cuff of claim 24 further comprising:
a controller operable to selectively pressurize the pressurizing device;
a pressure sensor operable to indicate a pressure within either the first chamber or pressurizing device, the pressure sensor being in communication with the controller to provide information regarding the pressure of the material within the first chamber or the pressurizing device to the controller.

29. The cuff of claim 24, further comprising a microphone positioned adjacent to the first chamber and between the pressurizing device and the body for detecting acoustic signals generated by the body.

30. The cuff of claim 24, further comprising a hydrophone coupled to the first chamber for detecting signals coupled to the first chamber.

31. The cuff of claim 25, wherein signals indicative of the gas pressure within the second chamber are converted by the gas pressure sensor and provided as a signal to the controller.

32. The cuff of claim 28, wherein signals indicative of the pressure within the first chamber are converted by the pressure sensor and provided as a signal to the controller.

33. The cuff of claim 32, wherein the pressure sensor has a sufficiently fast response rate such that it is capable of detecting pressure signals within the body and pressure oscillation signals from the first chamber.

34. The cuff of claim 28, further comprising a plurality of filters for separating the liquid pressure signals, the pressure oscillation signals and the Korotkoff sounds.

35. The cuff of claim 28, further comprising an electrical interface between said cuff and external electronics, wherein the interface is one of wired and wireless.

36. The cuff of claim 25, wherein the first chamber and the second chamber are formed as a unitary structure with a common wall separating the first chamber and the second chamber.

37. The cuff of claim 24, further comprising a tilt sensor for indicating a relative positioning between the cuff and a portion of the body.

38. The cuff of claim 24, further comprising a support structure positioned on an exterior side of the second chamber.

39. The cuff of claim 24, wherein the first chamber is formed of a material which includes a pliant and flexible material.

40. The cuff of claim 24, wherein the first chamber is formed of latex or polyethylene.

41. The cuff of claim 24, wherein the first chamber includes a peripheral portion having a thickness which is greater than a thickness of a central portion of the first chamber.

42. The cuff of claim 24, wherein the first chamber includes a back wall having a thickness which is greater than a thickness of a peripheral portion of the first chamber.

43. The cuff of claim 24, wherein the first chamber includes a back wall having a thickness which is greater than a thickness of a front wall of the first chamber.

44. The cuff of claim 24, wherein the first chamber is molded as a flat pliant or flexible plate.

45. The cuff of claim 24, wherein the first chamber is pre-shaped to conform to a shape of the body.

46. The cuff of claim 25 further comprising a pressurizing device for use in selectively pressurizing the gas of the second chamber.

47. The cuff of claim 24, wherein the pressurizing device includes electrically activated polymer, an air tank, or a pressurized gas cartridge.

48. The cuff of claim 24, wherein the non-compressible material comprises material having a density which is substantially within 50% of a density of human blood.

49. The cuff of claim 24, wherein the non-compressible material comprises material selected from the group consisting of water, mineral oil, aqueous gel, and organic gel.

50. A pressurizing cuff for applying pressure to a portion of the human body and detecting signals indicative of arterial pressure from the body, the cuff comprising:
a first chamber containing a substantially non-compressible material, the first chamber having a first side positioned adjacent the human body and a second, opposite side;
a second chamber containing a gas, said second chamber being positioned adjacent the second side of the first chamber, such that said first chamber is positioned between the human body and said second chamber;
a pressurizing device for selectively pressurizing and depressurizing the gas of the second chamber such that pressure is selectively applied by the second chamber to the first chamber, which in turn applies pressure to an artery within the human body;
a controller operable to selectively control the pressurizing device to pressurize the gas of the second chamber;
a pressure sensor operable to indicate a pressure of the material within the first chamber, the pressure sensor being in communication with the controller to provide information regarding the pressure of the material within the first chamber to the controller;
an electronics analysis module for using the information regarding the pressure of the material within the first chamber to thereby indicate a systolic and diastolic pressure of the human body.

51. The cuff of claim 50, wherein the pressure sensor has a sufficiently fast response rate such that it is capable of detecting pressure signals within the body and pressure oscillation signals from the first chamber.

52. The cuff of claim 50, wherein the first chamber and the second chamber are formed as a unitary structure with a common wall separating the first chamber and the second chamber.

53. The cuff of claim 50, further comprising a position sensor for indicating a relative positioning between the cuff and a portion of the body.

54. The cuff of claim 50, further comprising a support structure positioned on an exterior side of the second chamber.

55. The cuff of claim 50, wherein the first chamber is formed of a material which includes a pliant and flexible material.

56. The cuff of claim 50, wherein the first chamber includes a peripheral portion having a thickness which is greater than a thickness of a central portion of the first chamber.

57. The cuff of claim 50, wherein the non-compressible material comprises material having a density which is substantially within 50% of a density of human blood.

58. A method for detecting blood pressure of a human body using a pressurizing cuff, the method comprising the following steps:
placing a pressurizing cuff adjacent a portion of the human body and detecting signals indicative of arterial pressure from the body, the cuff including
a first chamber containing a substantially non-compressible material, the first chamber having a first side positioned adjacent the human body and a second, opposite side;
a second chamber containing a gas, said second chamber being positioned adjacent the second side of the first chamber, such that said first chamber is positioned between the human body and said second chamber;
using the second chamber as a pressurizing device to selectively pressurize and depressurize the gas that pressure is selectively applied by the second chamber to the first chamber, which in turn applies pressure to an artery within the human body;
using a controller to selectively control the pressurizing device to pressurize the gas of the second chamber;
using a pressure sensor to indicate a pressure of the material within the first chamber, the pressure sensor being in communication with the controller to provide information regarding the pressure of the material within the first chamber to the controller;
using an electronics analysis module to analyze the information regarding the pressure of the material within the first chamber to thereby indicate a systolic and diastolic pressure of the human body.
